(19) United States
(12) Patent Application Publication
(10) Pub. No.: US 2004/0235120 A1
(43) Pub. Date: Nov. 25, 2004

(54) Method for producing vitamin b12

(76) Inventors: Andreas Kunkel, Neustadt (DE); Jan-Henning Martens, Braunschweig (DE); Dieter Jahn, Wolfenbuttel (DE); Heiko Barg, Braunschweig (DE); Martin Warren, Berkshire (GB)

Correspondence Address: KEIL & WEINKAUF, 1350 CONNECTICUT AVENUE, N.W., WASHINGTON, DC, 20036, US

(21) Appl. No.: 10/487,202

(22) PCT Filed: Aug. 10, 2002
(86) PCT No.: PCT/EP02/09272

(30) Foreign Application Priority Data
Aug. 22, 2001 (DE) 101 41 132.4
Oct. 11, 2001 (DE) 101 50 359.8

(51) Int. Cl. C12P 19/30; C07H 21/04; C12N 1/21

(52) U.S. Cl. 435/89; 435/252.31; 536/23.2

(57) ABSTRACT

The present invention relates to a process for preparing vitamin B12 using Bacillus megaterium.

The present invention relates to a process for preparing vitamin B12 using Bacillus megaterium.

As long ago as the third decade of this century, vitamin B₁₂ was discovered indirectly through its effect on the human body by George Minot and William Murphy (Stryer, L., 1988, in Biochemie, fourth edition pp. 528-531, Spektrum Akademischer Verlag GmbH, Heidelberg, Berlin, N.Y.). Vitamin B₁₂ was purified and isolated for the first time in 1948, so that only eight years later, in 1956, its complex three-dimensional crystal structure was elucidated by Dorothy Hodgkin (Hodgkin, D. C. et al., 1956, Structure of Vitamin B₁₂. Nature 176, 325-328 and Nature 178, 64-70). The naturally occurring final products of the biosynthesis of vitamin B₁₂ are 5′-deoxyadenosylcobalamin (coenzyme B₁₂) and methylcobalamin (MeCbl), while vitamin B₁₂ is defined as cyanocobalamin (CNCbl) which is the form which is principally prepared and dealt with by industry. In the present invention, unless specifically stated, vitamin B₁₂ always refers to all three analogous molecules.

The species B. megaterium was described for the first time by De Bary more than 100 years ago (1884). Although generally classified as a soil bacterium, B. megaterium can also be detected in various other habitats such as seawater, sediments, rice, dried meat, milk or honey. It is often associated with pseudomonads and actinomyces. B. megaterium is, like its close relation Bacillus subtilis, a Gram-positive bacterium and is distinguished inter alia by its relatively distinct size, which gives it its name, of 2×5 μm, a G+C content of about 38% and a very pronounced sporulation ability. Even miniscule amounts of manganese in the growth medium are sufficient for this species to carry out complete sporulation, an ability which is comparable only with the sporulation efficiency of some thermophilic bacilli. Because of its size and its very efficient sporulation and germination, diverse investigations have been carried out in the molecular bases of these processes in B. megaterium, so that more than 150 B. megaterium genes involved in its sporulation and germination have now been described. Physiological investigations on B. megaterium (Priest, F. G. et al., 1988, A Numerical Classification of the Genus Bacillus, J. Gen. Microbiol. 134, 1847-1882) classified this species as an obligately aerobic, spore-forming bacterium which is urease-positive and Voges-Proskauer negative and is unable to reduce nitrate. One of the most prominent properties of B. megaterium is its ability to utilize a large number of carbon sources. Thus it utilizes a very large number of sugars and has been found, for example, in corn syrup, waste from the meat industry and even in petrochemical waste. In relation to this ability to metabolize an extremely wide range of carbon sources, B. megaterium can be equated without restriction with the pseudomonads (Vary, P. S., 1994, Microbiology, 40, 1001-1013, Prime time for Bacillus megaterium).

The advantages of the wide use of B. megaterium in the industrial production of a wide variety of enzymes, vitamins etc. are manifold. These include, firstly and certainly, the circumstance that plasmids transformed into B. megaterium prove to be very stable. This must be viewed in direct connection with the possibility which has now been established of transforming this species for example by polyethylene glycol treatment. Until a few years ago, this was still a major impediment to the use of B. megaterium as producer strain. The advantage of relatively well developed genetics must also be regarded in parallel with this, being exceeded within the Bacillus genus only by B. subtilis. Secondly, B. megaterium has no alkaline proteases, so that scarcely any degradation has been observed on production of heterologous proteins. It is additionally known that B. megaterium efficiently secretes products of commercial interest, as is utilized for example in the production of α- and β-amylase. In addition, the size of B. megaterium makes it possible to accumulate a large biomass before excessive population density leads to death. A further favorable circumstance of very great importance in industrial production using B. megaterium is the fact that this species is able to prepare products of high value and very high quality from waste and low-quality materials. This possibility of metabolizing an enormously wide range of substrates is also reflected in the use of B. megaterium as soil detoxifier able to break down even cyanides, herbicides and persistent pesticides. Finally, the fact that B. megaterium is completely apathogenic and produces no toxins is of very great importance, especially in the production of foodstuffs and cosmetics. Because of these many advantages, B. megaterium is already employed in a large number of industrial applications such as the production of α- and β-amylase, penicillin amidase, the processing of toxic waste or aerobic vitamin B₁₂ production (summarized in Vary, P. S., 1994, Microbiology, 40, 1001-1013, Prime time for Bacillus megaterium).

The use of Bacillus megaterium is of great economic interest because it has a number of advantages for use in the biotechnological production of various products of industrial interest. In the large-scale industrial fermentation of aerobic microorganisms, however, problems regularly arise, especially with an efficient oxygen supply to the bacterial cultures, which are associated with considerable losses of product yield.

It is an object of the present invention to optimize the preparation of vitamin B12 using Bacillus megaterium.

We have found that this object is achieved by a process for preparing vitamin B12 using a culture containing Bacillus megaterium, in which the fermentation is carried out under anaerobic conditions.

It is possible in principle to employ for the purposes of the present invention all usual B. megaterium strains suitable as vitamin B12 producer strains.

Vitamin B12 producer strains mean for the purposes of the present invention Bacillus megaterium strains or homologous microorganisms which have been altered by classical and/or molecular genetic methods so that their metabolic flux is increased in the direction of the biosynthesis of vitamin B12 or its derivatives (metabolic engineering). For example, one or more gene(s) and/or the corresponding enzymes in these producer strains which are located at key positions in the metabolic pathway which are crucial and subject to correspondingly complex regulation (bottleneck) have their regulation modified or are even deregulated. The present invention encompasses in this connection all previously known vitamin B12 producer strains, preferably of the genus Bacillus or homologous organisms. The strains which are advantageous according to the invention include, in particular, the strains DSMZ 32, DSMZ 509 and DSMZ 2894 of B. megaterium.

It has been possible to show according to the invention that B. megaterium is capable of living anaerobically, and, in addition, vitamin B12 production is higher under these conditions than under aerobic conditions. Comparison of vitamin B12 production by B. megaterium under aerobic and anaerobic growth conditions shows clearly that anaerobic vitamin B12 production is increased in all the strains investigated by a factor of at least 3 to 4 compared with aerobic vitamin B12 production. Further increases can be achieved by systematic optimization of the growth conditions and of the composition of the culture medium, and of the bacterial strains employed.

It is possible according to the invention for the preparation of vitamin B12 using Bacillus megaterium to be increased for example by adding at least cobalt to the culture medium. The addition of, for example, betaine, methionine, glutamate, dimethylbenzimidazole or choline or combinations thereof also have advantageous effects on vitamin B12 production using the process of the invention. Combination of the aforementioned compounds, singly or combinations thereof, with cobalt may also be advantageous.

The present invention accordingly also relates to a process which is distinguished by addition of at least cobalt to the culture medium. That is to say, cobalt can be added for example singly or in combination with at least betaine, methionine, glutamate, dimethylbenzimidazole or choline or combinations of the last-mentioned compounds. In one variant of the process of the invention, the vitamin B-12 content can be increased by adding from about 200 to 750 μm, preferably 250 to 500 μm, cobalt per liter of culture medium.

A further variant of the present invention encompasses a process of the aforementioned type in which B. megaterium is fermented initially aerobically and then anaerobically. In an advantageous variant, the transition from aerobic to anaerobic fermentation takes place in the exponential growth phase of the aerobically fermented cells. It is advantageous in this connection for the transition from aerobic to anaerobic fermentation to take place in the middle or at the end, preferably at the end, of the exponential growth phase of the aerobically growing cells.

In this connection, preference is given according to the invention to a process in which the transition from aerobic to anaerobic fermentation takes place as soon as the aerobic culture has reached its maximum optical density, but at least an optical density of about 2 to 3.

Anaerobic conditions mean, both in the single stage and in the two-stage process of the invention, for the purposes of the present invention those conditions which occur when the bacteria are transferred after aerobic culture into anaerobic bottles and fermented there. The time of transfer into the anaerobic bottles takes place, especially in the two-stage process, as soon as the aerobically cultured bacterial cells are in the exponential growth phase. This means that, after transfer into the anaerobic bottles, the bacteria consume the oxygen present therein, and no further oxygen is supplied. These conditions may also be referred to as semi-anaerobic. Corresponding procedures are conventional laboratory practice and are known to the skilled worker.

Comparable conditions also prevail when the bacteria are initially cultivated aerobically in a fermentor and then the oxygen supply is gradually reduced so that semi-anaerobic conditions are eventually set up.

In a special variant of the present invention it is also possible for example to create strictly anaerobic conditions by adding reducing agents to the culture medium.

It is not absolutely necessary in general for a fermentation of the invention under anaerobic conditions (whether semi-anaerobic or strictly anaerobic) for the bacteria to be cultured aerobically (preculture). This means that the bacteria can also be cultured under anaerobic conditions and then be fermented further under semi-anaerobic or strictly anaerobic conditions. It is also conceivable for the inoculum to be taken directly from strain maintenance and employed for preparing vitamin B12 under anaerobic conditions

In one variant of the present invention, the fermentation takes place under aerobic conditions with addition of about 250 μm cobalt; under anaerobic conditions, addition of about 500 μm cobalt is advantageous.

Also included according to the invention are genetically modified bacterial strains which can be produced by classical mutagenesis or targeted molecular biology techniques and appropriate selection methods. Starting points of interest for targeted genetic manipulation are, inter alia, points where the biosynthetic pathways leading to vitamin B-12 branch, through which the metabolic flux can be deliberately guided in the direction of maximum vitamin B₁₂ production. Targeted modifications of genes involved in the regulation of the metabolic flux also include investigations and alterations of the regulatory regions upstream and downstream of the structural genes, such as, for example, the optimization and/or the exchange of promoters, enhancers, terminators, ribosome binding sites etc. The invention also encompasses improving the stability of the DNA, mRNA or of the proteins encoded by them, for example by reducing or preventing degradation by nucleases or proteases.

The present invention relates to the corresponding nucleotide sequences coding for enzymes involved in the biosynthesis of vitamin B12. The present invention relates in particular also to an isolated nucleotide sequence coding for enzymes involved in the biosynthesis of uroporphyrinogen III, organized in the hemAXCDBL operon from B. megaterium having a nucleotide sequence as shown in SEQ ID No. 1 or alleles thereof. Also included according to the invention are the enzymes which are encoded by the hemAXCDBL operon from B. megaterium and have the amino acid sequences shown in SEQ ID No. 2-6, and isoenzymes or modified forms thereof.

Isoforms mean enzymes having the same or comparable substrate specificity and specificity of action but having a different primary structure.

Modified forms mean according to the invention enzymes in which alterations in the sequence are present, for example at the N and/or C terminus of the polypeptide or in the region of conserved amino acids, but without impairing the function of the enzyme. These changes can be carried out in the form of amino acid exchanges by methods known per se.

A particular embodiment of the present invention encompasses variants of the polypeptides of the invention whose activity is weakened or strengthened, for example by amino acid exchanges, compared with the respective initial protein. The same applies to the stability of the enzymes of the invention in the cells which have, for example, increased or diminished susceptibility to degradation by proteases.

An isolated nucleic acid or an isolated nucleic acid fragment means according to the invention a polymer of RNA or DNA which may be single- or double-stranded and may optionally comprise natural, chemically synthesized, modified or artificial nucleotides. The term DNA polymer also includes in this connection DNA, cDNA or mixtures thereof.

Alleles mean according to the invention functionally equivalent nucleotide sequences, i.e. those having essentially the same effect. Functionally equivalent sequences are sequences which, despite a differing nucleotide sequence, still have the desired functions for example through the degeneracy of the genetic code. Functional equivalents thus encompass naturally occurring variants of the sequences described herein, and artificial nucleotide sequences which have, for example, been obtained by chemical synthesis and, where appropriate, adapted to the codon usage of the host organism. Functionally equivalent sequences additionally encompass those having an altered nucleotide sequence which confers on the enzyme for example asensitivity or resistance to inhibitors.

A functional equivalent means in particular also natural or artificial mutations of an originally isolated sequence which continue to show the desired function. Mutations comprise substitutions, additions, deletions, transpositions or insertions of one more nucleotide residues. These include so-called sense mutations which may, at the protein level, lead for example to exchange of conserved amino acids but which do not lead to a fundamental change in the activity of the protein and are thus functionally neutral. This also includes changes in the nucleotide sequence which, at the protein level, affect the N or C terminus of a protein with, however, negligible impairment of the function of the protein. These changes may in fact exert a stabilizing effect on the protein structure.

The present invention also encompasses for example those nucleotide sequences which are obtained by modifying the nucleotide sequence, resulting in corresponding derivatives. The aim of such a modification may be, for example, further localization of the coding sequence present therein, or, for example, insertion of further restriction enzyme cleavage sites. Functional equivalents are also variants whose function has been weakened or strengthened compared with the initial gene or gene fragment.

The present invention additionally relates to artificial DNA sequences as long as they confer, as described above, the desired properties. Such artificial DNA sequences can be found, for example, by back-translation of proteins constructed by computer-assisted programs (molecular modeling) or by in vitro selection. Coding DNA sequences which have been obtained by back-translation of a polypeptide sequence according to the codon usage specific for the host organism are particularly suitable. The specific codon usage can easily be found by a skilled worker familiar with methods of molecular genetics by computer analyses of other, previously known genes of the organism which is to be transformed.

Homologous sequences mean according to the invention those which are complementary to the nucleotide sequences of the invention and/or hybridize with the latter. The term hybridizing sequences includes according to the invention substantially similar nucleotide sequences from the group of DNA or RNA which enter into a specific interaction (binding) with the nucleotide sequences mentioned previously under stringent conditions known per se. These also include short nucleotide sequences with a length of, for example, 10 to 30, preferably 12 to 15, nucleotides This also encompasses according to the invention inter alia so-called primers or probes.

The invention also includes the sequence regions preceding (5- or upstream) and/or following (3′ or downstream) the coding regions (structural genes). These sequence regions include in particular those having a regulatory function. They may influence the transcription, the RNA stability or the RNA processing, and the translation. Examples of regulatory sequences are, inter alia, promoters, enhancers, operators, terminators or translation enhancers.

The present invention further emcompasses a gene structure comprising the isolated nucleotide sequence of the aforementioned type or parts thereof, and nucleotide sequences with regulatory function operatively linked thereto.

An operative linkage means the sequential arrangement for example of promoter, coding sequence, terminator and, where appropriate, further regulatory elements in such a way that each of the regulatory elements is able to carry out its proper function in the expression of the coding sequence. These regulatory nucleotide sequences may be of natural origin or be obtained by chemical synthesis. A suitable promoter is in principle any promoter which is able to control gene expression in the appropriate host organism. A possibility for this according to the invention is also a chemically inducible promoter able to control the expression of the genes subject to it in the host cell to a particular time. The β-galacosidase or arabinose system may be mentioned here by way of example.

A gene structure is produced by fusing a suitable promoter with at least one nucleotide sequence of the invention by conventional techniques of recombination and cloning as described, for example, in Sambrook, J. et al., 1989, In Molecular cloning; a laboratory manual. 2^{nd} Ed., Cold Spring Harbor Laboratory Press, Cold Spring Harbor, N.Y.

Adaptors or linkers can be attached to the fragments for the joining together of the DNA fragments.

The invention also encompasses a vector comprising an isolated nucleotide sequence of the aforementioned type or parts thereof or a gene structure of the aforementioned type, and additional nucleotide sequences for selection, replication in the host cell and/or integration into the host cell genome. Numerous examples of such additional sequences are described in the literature and are not mentioned further.

Suitable systems for the transformation and overexpression of the genes mentioned in B megaterium are, for example, the plasmids pWH1510 and pWH1520, and the plasmid-free overexpression strain B. megaterium WH320, which are described by Rygus, T. et al. (1991, Inducible high level expression of heterologous genes in Bacillus megaterium, Appl. Microbiol. and Biotechnol., 35, 5: 594-599). The plasmids are also commercially available (Q biogens and MoBiTec). However, the systems mentioned are not limiting for the present invention.

The present invention also relates to a process which is distinguished by fermentation of a Bacillus megaterium strain whose hemAXCDBL operon or parts thereof shows/show enhanced expression.

A variant of the present invention also encompasses a process in which there is fermentation of a genetically altered Bacillus megaterium strain whose hemAXCDBL operon or parts thereof is/are present in increased copy number in the cell compared with the not genetically altered Bacillus. The number of copies may vary between 2 to several hundred.

Increased gene expression (overexpression) can be achieved by increasing the copy number of the appropriate genes. A further possibility is to alter the promoter region and/or regulatory region and/or the ribosome binding site located upstream from the structural gene appropriately in such a way that expression takes place at an increased rate. Expression cassettes incorporated upstream from the structural gene act in the same way. It is additionally possible to increase the expression during vitamin B12 production by inducible promoters.

Expression is likewise improved by measures to extend the life span of the mRNA. The genes or gene constructs may either be present in plasmids in varying copy number and/or be integrated and/or amplified in the chromosome.

A further possibility is also for the activity of the enzyme itself to be increased or be enhanced by preventing breakdown of the enzyme protein.

A further possible alternative for achieving overexpression of the relevant genes is by altering the composition of the media and management of the culture.

The present invention further relates to a transformed Bacillus megaterium strain for use in a process for vitamin B12 production of the aforementioned type, which shows enhanced expression and/or increased copy number of the nucleotide sequence of the hemAXCDBL operon or parts thereof.

Also included according to the invention is a transformed Bacillus megaterium strain which comprises, in replicating form, a gene structure or a vector of the aforementioned type.

The present invention further relates to the use of the isolated nucleotide sequence of the hemAXCDBL operon or parts thereof or of the gene structure or a vector of the aforementioned type for producing a transformed Bacillus megaterium strain of the aforementioned type. The present invention also includes the use of the transformed Bacillus megaterium strain of the aforementioned type for producing vitamin B12.

The following examples serve to illustrate the present invention. However, they do not have a limiting effect on the invention.

1. Chemicals and Molecular Biology Agents

| | |
|---|---|
| DNA isolation kt | Qiagen |
| Fast-Link ligation kit | Biozym |
| Molecular biological enzymes | Amersham-Pharmacia, NEN-LifeScience |
| Growth media | Difco |

2. Bacterial Strains and Plasmids

All the bacterial strains and plasmids used in this work are listed in tables 1 and 2.

3. Buffers and Solutions

3.1. Minimal medium

| | | |
|---|---|---|
| E. coli minimal medium | | |
| K₂HPO₄ | 60.3 | mM |
| KH₂PO₄ | 33.1 | mM |
| (NH₄)₂SO₄ | 7.6 | mM |
| Sodium citrate | 1.7 | mM |
| MgSO₄ | 1.0 | mM |
| D-Glucose | 10.1 | mM |
| Thiamine | 3.0 | μM |
| Casamino acids | 0.025 | % (w/v) |
| 15 g/l agar-agar were added for solid media. | | |
| Mopso minimal medium | | |
| Mopso (pH 7.0) | 50.0 | mM |
| Tricine (pH 7.0) | 5.0 | mM |
| MgCl₂ | 520.0 | μM |
| K₂SO₄ | 276.0 | μM |
| FeSO₄ | 50.0 | μM |
| CaCl₂ | 1.0 | mM |
| MnCl₂ | 100.0 | μM |
| NaCl | 50.0 | mM |
| KCl | 10.0 | mM |
| K₂HPO₄ | 1.3 | mM |
| (NH₄)₆Mo₇O₂₄ | 30.0 | pM |
| H₃BO₃ | 4.0 | nM |
| CoCl₂ | 300.0 | pM |
| CuSO₄ | 100.0 | pM |
| ZnSO₄ | 100.0 | pM |
| D-Glucose | 20.2 | mM |
| NH₄Cl | 37.4 | mM |
| Titration reagent was KOH solution. | | |
| S. typhimurium minimal medium | | |
| NaCl | 8.6 | mM |
| Na₂HPO₄ | 33.7 | mM |
| KH₂PO₄ | 22.0 | mM |
| NH₄Cl | 18.7 | mM |
| D-Glucose | 20.2 | mM |
| MgSO₄ | 2.0 | mM |
| CaCl₂ | 0.1 | mM |
| 15 g/l agar-agar were added for solid media. | | |

3.2. Solutions for protoplast transformation of B. megaterium

| | | |
|---|---|---|
| SMMP buffer | | |
| Antibiotic Medium No. 3 (Difco) | 17.5 | g/l |
| Sucrose | 500.0 | mM |
| Na maleate (pH 6.5) | 20.0 | mM |
| MgCl₂ | 20.0 | mM |
| Titration reagent was NaOH solution. | | |
| PEG-P solution | | |
| PEG 6000 | 40.0 | % (w/v) |
| Sucrose | 500.0 | mM |
| Na maleate (pH 6.5) | 20.0 | mM |
| MgCl₂ | 20.0 | mM |
| Titration reagent was NaOH solution. | | |
| cR5 top agar | | |
| Sucrose | 300.0 | mM |
| Mops (pH 7.3) | 31.1 | mM |
| NaOH | 15.0 | mM |
| L-Proline | 52.1 | mM |
| D-Glucose | 50.5 | mM |
| K₂SO₄ | 1.3 | mM |
| MgCl₂ × 6 H₂O | 45.3 | mM |
| KH₂PO₄ | 313.0 | μM |
| CaCl₂ | 13.8 | mM |
| Agar-agar | 4.0 | g/l |
| Casamino acids | 0.2 | g/l |
| Yeast extract | 10.0 | g/l |
| Titration reagent was NaOH solution. | | |

4. Media and Additions to Media

4.1 Media

Unless stated specially, the Luria-Bertani Broth (LB) complete medium as described in Sambrook, J. et al. (1989, In Molecular cloning; a laboratory manual. 2^{nd} Ed., Cold Spring Harbor Laboratory Press, Cold Spring Harbor, N.Y.) was used.

4.2. Additions

Additions such as carbon sources, amino acids or antibiotics were either added to the media and autoclaved together or made up as concentrated stock solution in water and sterilized, where appropriate by filtration. The substances were added to the media which had been autoclaved and cooled to below 50° C. With substances sensitive to light, such as tetracycline, care was taken to incubate in the dark. The final concentrations normally used were as follows:

| | | |
|---|---|---|
| Ampicillin | 296 | μM |
| Tetracycline | 21 | μM |
| ALA | 298 | μM |
| Heme | 153 | μM |
| X-Gal | 98 | μM |
| Methionine | 335 | μM |
| Cysteine | 285 | μM |
| Sodium nitrate | 10 | mM |
| Sodium nitrite | 10 | mM |
| Disodium fumarate | 10 | mM |
| Glucose | 10 | mM |
| Ammonium chloride | 37 | mM |
| Xylose | 33 | mM |
| Lysozyme | 10 | μg/ml |
| Casamino acids | 0.025 | % (w/v) |

5. Microbiological Techniques

5.1. Sterilization

Unless indicated otherwise, all the media and buffers were steam-sterilized at 120° C. and a gage pressure of 1 bar for 20 min. Thermally sensitive substances were sterilized by filtration, and glassware was heat-sterilized at 180° C. for at least 3 h.

5.2. General Growth Conditions

Aerobic bacterial cultures were incubated in baffle flasks at 37° C. and at a minimum of 180 rpm. The incubation times were varied according to the desired optical densities of the bacterial cultures.

5.3. Conditions for Bacillus megaterium Growth

For the best possible aeration of aerobic cultures they were incubated in baffle flasks at 250 rpm and, unless indicated otherwise, at 30° C. Anaerobic cultures were fermented in a volume of 100 ml in small anaerobic bottles at 30° C. and 100 rpm. In both cases, care was taken to use media of constant quality, to inoculate in the ratio 1:100 from overnight cultures, and to use constant conditions for the overnight cultures.

5.4. Determination of the Cell Density

The cell density of a bacterial culture was determined by measuring the optical density at 578 nm, the assumption being that an OD₅₇₈ of one corresponds to a cell count of 1×09 cells.

5.5. Comparative Growth Investigations

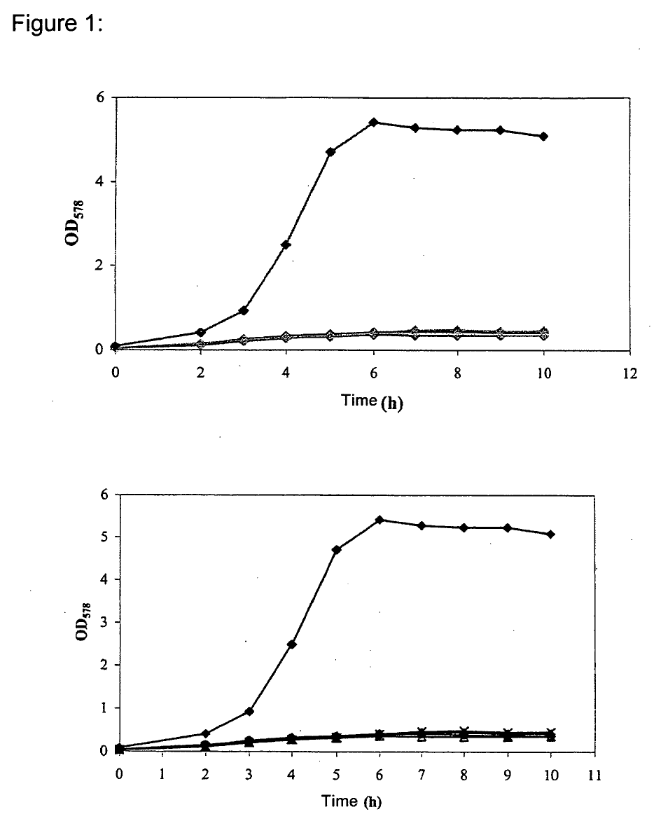

Comparative investigations of the aerobic and anaerobic growth behavior of various Bacillus megaterium strains were carried out and are depicted in FIGS. 1, 2, and 3. The strains employed are the strains B. megaterium DSMZ 32 (wild type) or the “producer strains” DSMZ 509 and DSMZ 2894. which are suitable for vitamin B12 production under aerobic conditions. However, the present invention is not limited to the use of these strains. Other strains suitable for the preparation of vitamin B12 are also conceivable, including genetically modified bacterial strains which can be produced by classical mutageneses or specific molecular biology techniques and appropriate selection methods.

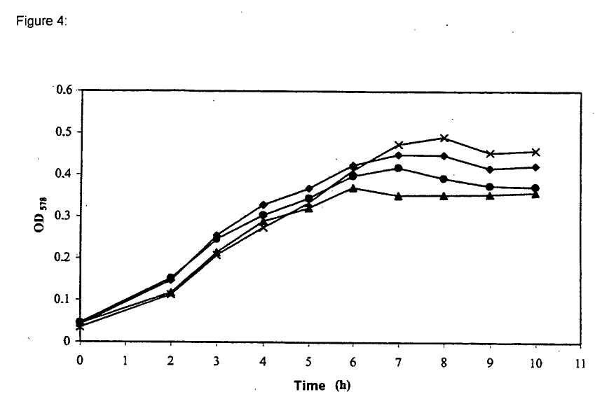

In the investigations of the extent to which B. megaterium is capable of anaerobic growth, the alternative electron acceptors nitrate, nitrite and fumarate were added in place of the aerobic electron acceptor oxygen to the culture medium (FIGS. 4-6). Parallel investigations were carried out into whether B. megaterium is also capable of fermentation in a medium without addition of these electron acceptors. It is clear in this connection from the results of the present invention that the electron acceptor nitrite exerts a toxic effect on bacterial growth. Fumarate also acts to inhibit growth. None of the added potential electron acceptors is able to stimulate anaerobic growth beyond the extent of the fermentative growth.

5.6. Comparative Investigations of Vitamin B12 Production

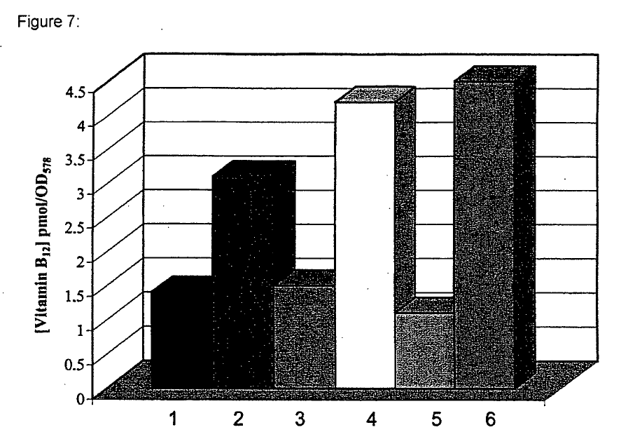

Analyses of vitamin B12 production under aerobic and anaerobic growth conditions for Bacillus megaterium were also carried out. The examples used according to the invention were the strains Bacillus megaterium DSMZ 32, DSMZ 509 and DSMZ 2894 under anaerobic conditions in glucose-containing (LB complete) medium, and the vitamin B12 content in pmol/OD₅₇₈ was measured at the end of the exponential growth phase. In parallel with this, the vitamin B12 production while living aerobically was investigated in the middle of the exponential growth phase. The results are shown in FIG. 7.

5.7. Effect of 5-aminolevulinic Acid (ALA) on Vitamin B12 Production

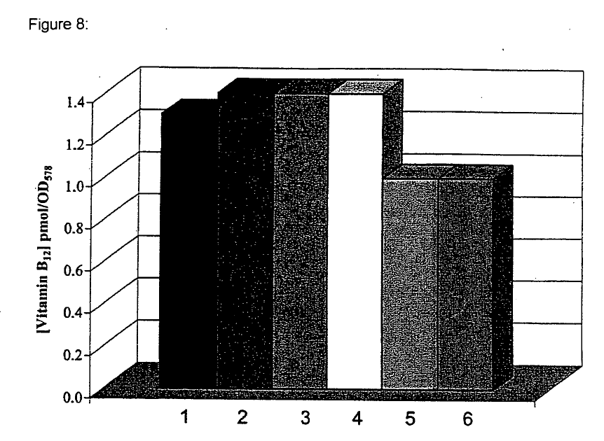

Since a central regulation point in the tetrapyrrole biosynthetic pathway for synthesizing vitamin B12 is the formation of 5-aminolevulinic acid (ALA), the effect of ALA on the vitamin B-12 production in Bacillus megaterium was investigated according to the invention. Aerobic vitamin B-12 production by B. megaterium with and without external addition of ALA is depicted in FIG. 8. ALA concentrations of about 50 μg/ml of culture medium were employed for this.

It was possible to show according to the invention that addition of ALA has no effect on vitamin B-12 production both on aerobic and on anaerobic fermentation of the bacterium. On this basis, regulation of vitamin B-12 biosynthesis appears not to be limited solely by the formation of ALA.

5.8. Quantitative Vitamin B₁₂ Analysis

Aerobic B. megaterium cultures were harvested in the middle of the exponential growth phase, and anaerobic cultures were harvested at the end of the exponential growth phase after determination of the OD₅₇₈ by centrifugation at 5 000 rpm for 15 minutes (Centrifuge 5403, Eppendorf). Washing with 40 ml of saline was followed by centrifugation again at 5 000 rpm for 15 min (Centrifuge 5403, Eppendorf). The resulting cell sediments were finally lyophilized. S. typhimurium metE cysG double mutants (Raux, E. et al., 1996, J. Bacteriol., 178: 753-767) were incubated on minimal medium containing methionine and cysteine at 37° C. overnight, scraped off the plate and washed with 40 ml of isotonic saline. After centrifugation, the cell sediment was resuspended in isotonic saline. The washed bacterial culture was cautiously mixed with 400 ml of cysteine-containing minimal medium agar at 47-48° C. 10 μl of the B. megaterium samples which had been resuspended in deionized sterile water and boiled in a water bath for 15 min were put onto the cold plates and incubated at 37° C. for 18 h. The diameters of the salmonella colonies which grow are then proportional to the vitamin B12 content of the B. megaterium samples applied. The vitamin B12 content in the investigated samples was deduced by comparison with a calibration plot constructed by adding 0.01, 0.1, 1, 10 and 40 pmol of vitamin B12. This standard method allows small amounts of vitamin B12 to be detected rapidly and very reproducibly in biological materials.

6. Molecular Biology Techniques

General techniques such as DNA preparation, restriction of DNA, ligation, PCR, sequencing, functional complementation, protein expression etc. form part of conventional laboratory practice and are described in Sambrook, J. et al. (1989, in Molecular cloning; a laboratory manual. 2^{nd} Ed., Cold Spring Harbor Laboratory Press, Cold Spring Harbor, N.Y.).

6.1. Protoplast Transformation of Bacillus megaterium

Protoplast Preparation:

50 ml of LB medium were inoculated with 1 ml of an overnight culture of B. megaterium and incubated at 37° C. At an OD₅₇₈ of 1, the cells were centrifuged at 15 000 rpm and 4° C. for 15 min (RC 5B Plus, Sorvall) and resuspended in 5 ml of SMMP buffer. After addition of lysozyme in SMMP buffer, the suspension was incubated at 37° C. for 60 min, and protoplast formation was checked under the microscope. After the cells had been harvested by centrifugation at 3 000 rpm and RT (Centrifuge 5403, Eppendorf), the cell sediment was cautiously resuspended in 5 ml of SMMP buffer, and the centrifugation step and washing step were carried out a second time. It was then possible after addition of 10% (v/v) glycerol, to divide the protoplast suspension into portions and freeze at −80° C.

Transformation:

500 μl of the protoplast suspension were mixed with 0.5 to 1 μg of DNA in SMMP buffer, and 1.5 ml of PEG-P solution were added. After incubation at RT for 2 min, 5 ml of SMMP buffer were added and, after cautious mixing, the suspension was centrifuged at 3 000 rpm and RT for 10 min (Centrifuge 5403, Eppendorf). The supernatant was removed immediately thereafter and the scarcely visible sediment was resuspended in 500 μl of SMMP buffer. The suspension was incubated shaking gently at 37° C. for 90 min. Then 50-200 μl of the transformed cells were mixed with 2.5 ml of cR5 top agar and put onto LB agar plates which contained antibiotics suitable for the selection. Transformed colonies were visible after incubation at 37° C. for two days.

6.2. Identification and Sequencing of the hemAXCDBL Operon From B. megaterium

The strategy chosen for cloning the hemAXCDBL operon was that of functional complementation of heme-auxotrophic E. coli mutants. For this purpose, a B. megaterium gene library was prepared by conventional methods. It was possible by functional complementation of the E. coli hemB mutant RP523 with this genomic B. megaterium plasmid gene library to isolate colonies which were again capable of complete tetrapyrrole biosynthesis, i.e. phenotypically represented the heme-prototrophic wild type. It was possible after plasmid DNA preparation and DNA sequencing of the vector-located B. megaterium DNA to identify an insert 3855 kb in size which codes for the hemAXCDBL operon sought. The corresponding nucleotide sequence is listed in SEQ ID No. 1, and the amino acid sequences derived therefrom are listed in SEQ ID No. 2-6.

The sequences upstream and downstream of the DNA fragment obtained by functional complementation were obtained using the Vectorette TM system from Sigma Genosis. The Turbo Pfu DNA polymerase from Strategene which, owing to its proofreading function, shows an extremely low rate of errors.

6.3. Transformation and Expression Systems

Suitable plasmids for transformation and overexpression of genes in B. megaterium are pWH1510 and pWH1520 and the plasmid-free overexpression strain B. megaterium WH320 (Rygus, T. et al., 1991, Inducible high level expression of heterologous genes in Bacillus megaterium, Appl. Microbiol. And Biotechnol., 35, 5: 594-599).

The control plasmid pWH1510 contains in the interrupted xylA reading frame a spoVG-lacZ fusion. SpoVG-lacZ refers in this case to the fusion of a very strong ribosome binding sequence of B. subtilis sporulation protein (spoVG) with the E. coli gene coding for β-galactosidase (lacZ). This plasmid is thus outstandingly suitable for investigating transformation efficiencies and overexpression conditions in B. megaterium.

The plasmid pWH1520 functions as the actual cloning and expression vector. Both vectors have a tetracycline resistance and an ampicillin resistance and the elements important for replication in E. coli and Bacillus spp. They are thus amenable to all techniques established in E. coli for derivatives of the plasmid pBR322. Both vectors contain the B. megaterium xylA and xylR genes of the xyl operon with their regulatory sequences (Rygus, T. et al., 1991, Molecular Cloning, Structure; Promoters and Regulatory Elements for Transcription of the Bacillus megaterium Encoded Regulon for Xylose Utilization, Arch. Microbiol. 155, 535-542). XylA codes for xylose isomerase, while xylR codes for a regulatory protein which exerts strong transcriptional control on xylA. XylA is repressed in the absence of xylose. On addition of xylose there is an approximately 200-fold induction through derepression of xylA. A polylinker in the xylA reading frame makes it possible to fuse genes with xylA, which are then likewise under the strong transcriptional control of XyIR. It is moreover possible to choose between the alternatives of forming a transcription or translation fusion because the xylA reading frame is still completely intact upstream from the polylinker.

Key to the Figures

Bacterial strains and plasmids as shown in tables 1 and 2 were employed.

| | |
|---|---|
| Table 1: | Bacterial strains used |
| Table 2: | Plasmids used |

The present invention is explained further by means of the figures below.

FIG. 1 shows a comparison of the growth of B. megaterium DSMZ32 (wild type) under aerobic and anaerobic conditions at 30° C. Anaerobic growth was measured with addition of 10 mM nitrate (open diamonds), 10 mM nitrite (open triangles) and 10 mM fumarate (crosses). Fermentative (open circles) and aerobic growth (filled diamonds), took place in LB medium without additions. Samples were taken at the stated times, and the optical density at 578 nm was determined.

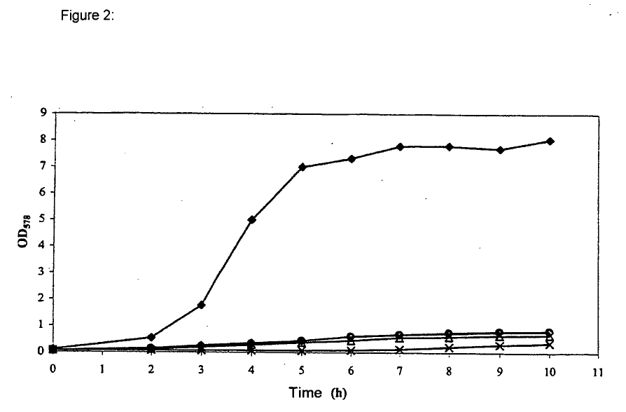

FIG. 2 shows a comparison of the growth of B. megaterium DSM509 under aerobic and anaerobic conditions at 30° C. Anaerobic growth was measured with addition of 10 mM nitrate (open diamonds), 10 mM nitrite (open triangles) and 10 mM fumarate (crosses). Fermentative (open circles) and aerobic growth (filled diamonds), took place in LB medium without additions. Samples were taken at the stated times, and the optical density at 578 nm was determined.

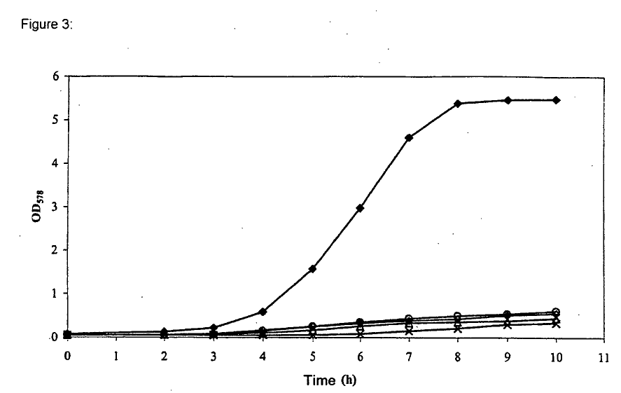

FIG. 3 shows a comparison of the growth of B. megaterium DSM2894 under aerobic and anaerobic conditions at 30° C. Anaerobic growth was measured with addition of 10 mM nitrate (open diamonds), 10 mM nitrite (open triangles) and 10 mM fumarate (crosses). Fermentative (open circles) and aerobic growth (filled diamonds), took place in LB medium without additions. Samples were taken at the stated times, and the optical density at 578 nm was determined.

FIG. 4 shows anaerobic growth of B. megaterium DSM32 (wild type) at 30° C. with addition of 10 mM nitrate (diamonds), 10 mM nitrite (triangles) and 10 mM fumarate (crosses). Fermentative growth (circles) took place in LB medium without additions. Samples were taken at stated times and the optical density at 578 nm was determined.

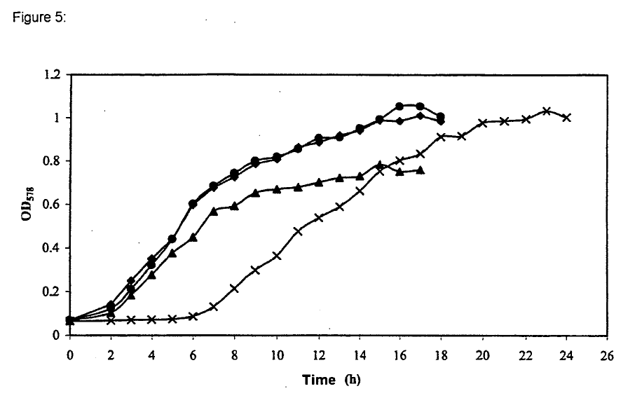

FIG. 5 shows anaerobic growth of B. megaterium DSM509 at 30° C. with addition of 10 mM nitrate (diamonds), 10 mM nitrite (triangles) and 10 mM fumarate (crosses). Fermentative growth (circles) took place in LB medium without additions. Samples were taken at stated times and the optical density at 578 nm was determined.

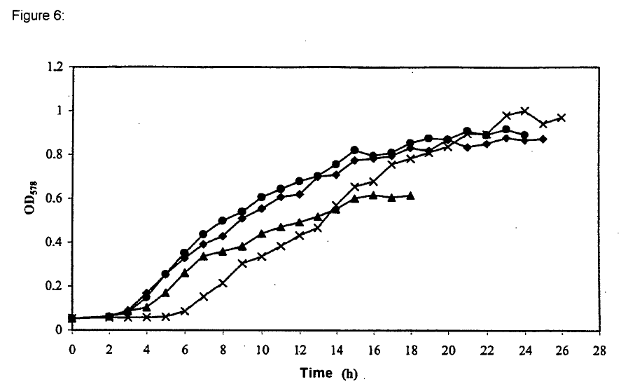

FIG. 6 shows anaerobic growth of B. megaterium DSM2894 at 30° C. with addition of 10 mM nitrate (diamonds), 10 mM nitrite (triangles) and 10 mM fumarate (crosses). Fermentative growth (circles) took place in LB medium without additions. Samples were taken at stated times and the optical density at 578 nm was determined.

FIG. 7 shows the vitamin B12 production by B. megaterium under aerobic and anaerobic growth conditions. The vitamin B12 content in the biomass has been indicated, determined in pmol/OD₅₇₈ for the wild-type strain B. megaterium DSM32 grown aerobically (1) and grown anaerobically (2), for B. megaterium DSM509 grown aerobically (3) and grown anaerobically (4), and for B. megaterium DSM2894 grown aerobically (5) and grown anaerobically (6).

FIG. 8 shows aerobic vitamin B₁₂ production by B. megaterium with and without external addition of 50 μg/ml ALA. The content of vitamin B₁₂ per biomass, measured in pmol/OD₅₇₈, was determined for the wild-type strain B. megaterium DSM32 without ALA addition (1), with ALA addition (2), for B. megaterium DSM509 without ALA addition (3), with ALA addition (4), and for B. megaterium DSM2894 without ALA addition (5) and with ALA addition (6).

We claim:

1. A process for preparing vitamin B12 using a culture comprising Bacillus megaterium, characterized in that the fermentation is carried out under anaerobic conditions.

2. A process as claimed in claim 1, characterized in that B. megaterium is fermented initially aerobically and then anaerobically.

3. A process as claimed in either of claims 1 or 2, characterized in that the transition from aerobic to anaerobic fermentation takes place in the exponential growth phase of the aerobically fermented cells.

4. A process as claimed in any of claims 1 to 3, characterized in that the transition from aerobic to anaerobic fermentation takes place in the middle or at the end, preferably at the end, of the exponential growth phase of the aerobically growing cells.

5. A process as claimed in any of claims 1 to 4, characterized in that at least cobalt is added to the culture medium.

6. A process as claimed in any of claims 1 to 5, characterized in that a Bacillus megaterium strain whose hemAXCDBL operon or parts thereof shows enhanced expression is fermented.

7. A process as claimed in any of claims 1 to 6, characterized in that a Bacillus megaterium strain whose hemAXCDBL operon is present in increased copy number in the cell is fermented.

8. An isolated nucleotide sequence coding for the enzymes which are involved in the biosynthesis of uroporphyrinogen III as shown in SEQ ID No. ID No. 2-6 or the isoforms thereof, organized in the hemAXCDBL operon having a nucleotide sequence as shown in SEQ ID No. 1 or alleles thereof.

9. A gene structure comprising the isolated nucleotide sequence as claimed claim 8 or parts thereof, and nucleotide sequences which are operatively linked thereto and have a regulatory function.

10. A vector comprising an isolated nucleotide sequence as claimed in claim 8 or parts thereof or a gene structure as claimed in claim 9, and additional nucleotide sequences for selection, replication in the host cell and/or integration into the host cell genome.

11. A transformed Bacillus megaterium strain for use in a process for vitamin B12 production as claimed in any of claims 1 to 7, characterized in that it shows enhanced expression and/or increased copy number of the nucleotide sequence as claimed in claim 8 or parts thereof.

12. A transformed Bacillus megaterium strain as claimed in claim 11, characterized in that it comprises, in replicating form, a gene structure as claimed in claim 9 or a vector as claimed in claim 10.

13. The use of the isolated nucleotide sequence as claimed in claim 8 or parts thereof or of the gene structure as claimed in claim 9 or of a vector as claimed in claim 10 for producing a transformed Bacillus megaterium strain as claimed in either of claims 11 or 12.

14. The use of the transformed Bacillus megaterium strain as claimed in either of claims 11 or 12 for producing vitamin B12.
